(19) United States
(12) Patent Application Publication
(10) Pub. No.: US 2018/0179574 A1
(43) Pub. Date: Jun. 28, 2018

(54) SYSTEMS AND METHODS FOR CHARACTERIZATION OF HYPERTRIGLYCERIDEMIA

(71) Applicant: The University of Chicago, Chicago, IL (US)

(72) Inventor: John B. Ancsin, Chicago, IL (US)

(21) Appl. No.: 15/757,074

(22) PCT Filed: Sep. 2, 2016
(86) PCT No.: PCT/US16/50178

§ 371 Date: Mar. 2, 2018

Related U.S. Application Data
(60) Provisional application No. 62/214,001, filed on Sep. 3, 2015.

(51) Int. Cl. C12Q 1/44

(52) U.S. Cl. CPC C12Q 1/44; G01N 2800/044; G01N 2333/92; C12Y 301/01034

(57) ABSTRACT

Provided herein are systems (e.g., reagents, devices, etc.) and methods for characterization of hypertriglyceridemia (HTG) in a subject. In particular, systems and methods, are provided for identifying the specific deficiency(ies) leading to HTG, and selecting an appropriate strategy for the treatment of HTG based thereof.

### CROSS REFERENCE TO RELATED APPLICATIONS

The present invention claims benefit of U.S. Provisional Patent Application 62/214,001, filed Sep. 3, 2015, which is incorporated by reference in its entirety.

### FIELD

Provided herein are systems (e.g., reagents, devices, etc.) and methods for characterization of hypertriglyceridemia (HTG) in a subject. In particular, systems and methods, are provided for identifying the specific deficiency(ies) leading to HTG, and selecting an appropriate strategy for the treatment of HTG based thereon.

### BACKGROUND

Triglyceride (a.k.a. triacylglyceride, TG) is a class of lipid composed of 3-fatty acids that are linked by ester bonds to glycerol. TG is the main constituent of vegetable oils and animal fats and in the blood, TG is transported primarily within TG-Rich Lipoproteins (TRLs), which include chylomicrons (˜84% TG), very low density lipoproteins (VLDL; ˜50% TG) and intermediate density lipoprotein (IDL; ˜31% TG). The impaired clearance of excess TG from blood is a major cause of clinical hypertriglyceridemia (HTG) (>3000−200 mg/dL, normal ˜150 mg/dL), a condition which affects about one-third of adults in the G7 countries (refs. 1,2; herein incorporated by reference in their entireties) and is a recognized risk factor for coronary artery disease (CAD) (including ischemic stroke, heart disease), metabolic syndrome, pancreatitis and cancer (refs. 3,4; herein incorporated by reference in their entireties). Familial hyperlipidemias are classified according to the Fredrickson classification where 5 of 6 categories involve deficiencies in the TG clearance pathway (ref. 5; herein incorporated by reference in its entirety). HTG may result from a number of causes, including lifestyle/dietetics, medications, and genetic abnormalities associated with one or more of the proteins involved in the regulation of plasma TG levels.

### SUMMARY

Provided herein are systems (e.g., reagents, devices, etc.) and methods for characterization of hypertriglyceridemia (HTG) in a subject. In particular, systems and methods, are provided for identifying the specific deficiency(ies) leading to HTG, and selecting an appropriate strategy for the treatment of HTG based thereon.

Chronically high concentrations of triglyceride (TG) in the blood is a condition known as hypertriglyceridemia (HTG) and is an important risk factor for a number of diseases, including coronary artery disease, diabetes and pancreatitis. HTG can be caused by the impaired function of one or more of the proteins normally responsible for the efficient clearance of excess TG from the blood. Provided herein are methods to identify the specific deficiency(ies) causing HTG (e.g., using routine blood samples from patients), and reagents, kits, and devices for carrying out such methods. In some embodiments, methods comprise at least two companion assays, one assessing the endogenous lipoprotein lipase (LpL) activity of blood leukocytes (white blood cells), and the second measuring the plasma's potential to stimulate LpL activity. The assays may be performed by any suitable techniques understood in the field (e.g., methods not toxic to cells). In some embodiments, one or both assays are performed using a quantitative fluorescent assay system (e.g., with a formulation which is nontoxic to cells and thus allows lipase determinations under physiological, pathological, or other relevant conditions).

In some embodiments, provided herein are methods of assessing lipoprotein lipase (LpL) activity in a subject, comprising: (a) exposing a blood sample from the subject to a triglyceride substrate, wherein the triglyceride substrate undergoes a detectable change upon hydrolysis by LpL; and (b) detecting the hydrolysis of the triglyceride substrate. In some embodiments, the triglyceride substrate is a fluorescent or colormetric triglyceride substrate which undergoes a change in fluorescence or color upon hydrolysis by LpL. In some embodiments, the triglyceride substrate is a fluorogenic triglyceride substrate (e.g., a triglyceride analog having a terminal fluorophore on one or more fatty acid chains). In some embodiments, the fluorogenic triglyceride substrate exhibits increased fluorescence upon hydrolysis by LpL. In some embodiments, the fluorogenic triglyceride substrate is a quenched substrate. In some embodiments, the LpL is circulating blood LpL. In some embodiments, the LpL does not comprise endothelial-bound LpL. In some embodiments, the blood sample and/or the subject have not been treated with heparin. In some embodiments, the blood sample is whole blood, a leukocyte-containing fraction of a fractioned blood sample, or purified leukocytes.

In some embodiments, provided herein are methods of assessing lipoprotein lipase (LpL) activity in a subject, comprising: (a) exposing a sample from the subject to a fluorogenic triglyceride substrate (e.g., a triglyceride analog having a terminal fluorophore on one or more fatty acid chains), wherein the fluorogenic triglyceride substrate undergoes a detectable increase in fluorescence intensity upon hydrolysis by LpL; and (b) detecting fluorescence. In some embodiments, the sample is an un-heparinized blood sample. In some embodiments, the sample comprises a blood fraction comprising leukocytes. In some embodiments, the LpL is circulating blood LpL. In some embodiments, the LpL does not comprise endothelial-bound LpL. In some embodiments, detecting fluorescence comprises measuring the fluorescence at a single timepoint. In some embodiments, detecting fluorescence comprises monitoring fluorescence over time.

In some embodiments, provided herein are methods of assessing the responsiveness of lipoprotein lipase (LpL) in a subject to apolipoproteins, comprising: (a) exposing a sample from the subject to an exogenous apolipoprotein; and (b) detecting hydrolysis of a triglyceride substrate (e.g., a triglyceride analog having a terminal fluorophore on one or more fatty acid chains). In some embodiments, the triglyceride substrate is a fluorescent or colormetric triglyceride substrate which undergoes a change in fluorescence or color upon hydrolysis by LpL. In some embodiments, the triglyceride substrate is a fluorogenic triglyceride substrate. In some embodiments, the fluorogenic triglyceride substrate exhibits increased fluorescence upon hydrolysis by LpL. In some embodiments, the fluorogenic triglyceride substrate is a quenched substrate. In some embodiments, the exogenous apolipoprotein comprises a known amount of an assay reagent comprising one or more apolipoproteins. In some embodiments, the exogenous apolipoprotein comprises apoC-II or an active peptide fragment thereof. In some embodiments, the exogenous apolipoprotein comprises apoA-V or an active peptide fragment thereof. In some embodiments, detecting fluorescence comprises measuring the fluorescence at a single timepoint. In some embodiments, detecting fluorescence comprises monitoring fluorescence over time. In some embodiments, the LpL is circulating blood LpL. In some embodiments, the LpL does not comprise endothelial-bound LpL. In some embodiments, the sample is a blood sample selected from the group consisting of whole blood, a leukocyte-containing fraction of a fractioned blood sample, or purified leukocytes. In some embodiments, the sample and/or the subject have not been treated with heparin. In some embodiments, methods further comprise comparing hydrolysis of the triglyceride substrate in the presence of exogenous apolipoprotein to a control value obtained in the absence of exogenous apolipoprotein.

In some embodiments, provided herein are methods of assessing the stimulating potential of a subject's apolipoprotein, comprising: (a) exposing a sample from the subject to a exogenous lipoprotein lipase (LpL) and a triglyceride substrate; and (b) detecting hydrolysis of the triglyceride substrate, wherein increased hydrolysis compared to a control of LpL and the triglyceride substrate indicates stimulation of LpL by the subject's apolipoproteins. In some embodiments, the sample comprises a blood sample selected from the group consisting of whole plasma, fractionated plasma, and isolated lipoproteins. In some embodiments, the sample does not comprise LpL. In some embodiments, the triglyceride substrate is a fluorescent or colormetric triglyceride substrate which undergoes a change in fluorescence or color upon hydrolysis by LpL. In some embodiments, the triglyceride substrate is a fluorogenic triglyceride substrate (e.g., a triglyceride analog comprising a terminal fluorophore on one or more fatty acids). In some embodiments, the fluorogenic triglyceride substrate exhibits increased fluorescence upon hydrolysis by LpL. In some embodiments, the fluorogenic triglyceride substrate is a quenched substrate.

In some embodiments, provided herein are methods, comprising: (a) measuring the basal lipoprotein lipase (LpL) activity in leukocytes from a sample from a subject; (b) contacting/incubating said leukocytes with apoC-II, and measuring the effect on LpL activity; (c) contacting/incubating said leukocytes with apoA-V, and measuring the effect on LpL activity; and (d) measuring the LpL stimulating potential of whole plasma and/or triglyceride-rich lipoproteins (TRLs) from said subject. In some embodiments, the sample is a whole blood sample or a processed blood sample. In some embodiments, methods further comprise separating plasma and/or TRLs from the leukocytes in the sample. In some embodiments, the LpL activity and the LpL stimulating potential are measured by a quantitative fluorescence assay system. In some embodiments, LpL activity and the LpL stimulating potential are measured by detecting the fluorescent degradation product of the hydrolysis of fluorogenic-TG analog by LpL. In some embodiments, stimulation of leukocytes is with peptides based on the apoC-II and/or apoA-V sequence (e.g., fragments of the apoC-II and/or apoA-V, or variants thereof).

In some embodiments, provided herein are methods comprising: (a) obtaining or receiving a blood sample from a subject; and (b) assaying two or more aspects of triglyceride hydrolysis, comprising: (i) measuring basal lipoprotein lipase (LpL) activity in leukocytes from the sample; (ii) contacting leukocytes from the sample with apoC-II or an active peptide fragment thereof, and measuring the effect on LpL activity; (iii) contacting leukocytes from the sample with apoA-V or an active peptide fragment thereof, and measuring the effect on LpL activity; and/or (iv) measuring the LpL stimulating potential of plasma and/or triglyceride-rich lipoproteins (TRLs) obtained from the sample. In some embodiments, methods comprise separating plasma and/or TRLs from the leukocytes in the sample. In some embodiments, the LpL activity and the LpL stimulating potential are measured by a quantitative fluorescence assay system.

In some embodiments, provided herein are methods of characterizing hypertriglyceridemia (HTG) in a subject, comprising: (a) measuring or having measured the basal lipoprotein lipase (LpL) activity, the effect of apoC-II (or apoC-II peptides) on LpL activity, and the effect of apoA-V (or apoA-V peptides) on LpL activity in leukocytes from the subject, and LpL stimulating potential of whole plasma and/or triglyceride-rich lipoproteins (TRLs) from said subject; and (b) classifying the subject according to one or more of the following criteria: (i) the subject does not suffer from HTG resulting from an apoprotein deficiency, LpL deficiency, or LpL response deficiency if: (A) said leukocytes exhibit basal LpL activity, (B) said leukocytes exhibit stimulation of LpL activity by apoC-II and apoA-V, and (C) said whole plasma and/or triglyceride-rich lipoproteins exhibit LpL stimulating potential; (ii) the subject suffers from HTG resulting from an LpL deficiency if: (A) said leukocytes do not exhibit basal LpL activity, (B) said leukocytes exhibit partial stimulation of LpL activity by apoC-II and/or apoA-V, and (C) said whole plasma and/or triglyceride-rich lipoproteins exhibit LpL stimulating potential; (iii) the subject suffers from HTG resulting from an apoprotein deficiency if: (A) said leukocytes exhibit basal LpL activity, (B) said leukocytes exhibit stimulation of LpL activity by apoC-II and apoA-V, and (C) said whole plasma and/or triglyceride-rich lipoproteins do not exhibit LpL stimulating potential; (iv) the subject suffers from HTG resulting from an LpL response deficiency if: (A) said leukocytes exhibit basal LpL activity, (B) said leukocytes exhibit stimulation of LpL activity by apoA-V but not apoC-II, and (C) said whole plasma and/or triglyceride-rich lipoproteins exhibit LpL stimulating potential; (v) the subject suffers from HTG resulting from an LpL response deficiency if: (A) said leukocytes exhibit basal LpL activity, (B) said leukocytes exhibit stimulation of LpL activity by apoC-II but not apoA-V, and (C) said whole plasma and/or triglyceride-rich lipoproteins exhibit LpL stimulating potential; (vi) the subject suffers from HTG resulting from overactive apoC-III if: (A) said leukocytes exhibit basal LpL activity, (B) said leukocytes do not exhibit stimulation of LpL activity by apoC-II or apoA-V, and (C) said whole plasma and/or triglyceride-rich lipoproteins exhibit LpL stimulating potential; and (vii) the subject suffers from HTG resulting from multiple causes if: (A) said leukocytes do not exhibit basal LpL activity, (B) said leukocytes exhibit partial stimulation of LpL activity by apoC-II and apoA-V, and (C) said whole plasma and/or triglyceride-rich lipoproteins do not exhibit LpL stimulating potential. In some embodiments, classifying the subject comprises describing the subject or a characteristic thereof in a report.

In some embodiments, provided herein are methods of characterizing hypertriglyceridemia (HTG) in a subject, comprising: (a) performing (or ordering) the lipase stimulating potential, lipase hydrolytic potential, and lipase response assays described herein; and (b) classifying or reporting the subject according to one or more of the following criteria: (i) the subject does not suffer from HTG resulting from an apolipoprotein deficiency, LpL deficiency, or LpL response deficiency if: (A) said leukocytes exhibit normal basal LpL activity, (B) said leukocytes exhibit stimulation of LpL activity by apoC-II and apoA-V, and (C) said whole plasma and/or triglyceride-rich lipoproteins exhibit LpL stimulating potential; (ii) the subject suffers from HTG resulting from an LpL deficiency if: (A) said leukocytes exhibit less than normal basal LpL activity, (B) said leukocytes exhibit partial stimulation of LpL activity by apoC-II and/or apoA-V, and (C) said whole plasma and/or triglyceride-rich lipoproteins exhibit LpL stimulating potential; (iii) the subject suffers from HTG resulting from an apoprotein deficiency if: (A) said leukocytes exhibit normal basal LpL activity, (B) said leukocytes exhibit stimulation of LpL activity by apoC-II and apoA-V, and (C) said whole plasma and/or triglyceride-rich lipoproteins do not exhibit LpL stimulating potential; (iv) the subject suffers from HTG resulting from an LpL response deficiency if: (A) said leukocytes exhibit normal basal LpL activity, (B) said leukocytes exhibit stimulation of LpL activity by apoA-V, and (C) said whole plasma and/or triglyceride-rich lipoproteins exhibit LpL stimulating potential; (v) the subject suffers from HTG resulting from an LpL response deficiency if: (A) said leukocytes exhibit basal LpL activity, (B) said leukocytes exhibit stimulation of LpL activity by apoC-II, and (C) said whole plasma and/or triglyceride-rich lipoproteins exhibit LpL stimulating potential; (vi) the subject suffers from HTG resulting from overactive apoC-III if: (A) said leukocytes exhibit normal basal LpL activity, (B) said leukocytes do not exhibit stimulation of LpL activity by apoC-II or apoA-V, and (C) said whole plasma and/or triglyceride-rich lipoproteins exhibit LpL stimulating potential; and (vii) the subject suffers from HTG resulting from multiple causes if: (A) said leukocytes exhibit less than normal basal LpL activity, (B) said leukocytes exhibit partial stimulation of LpL activity by apoC-II and apoA-V, and (C) said whole plasma and/or triglyceride-rich lipoproteins do not exhibit LpL stimulating potential.

In some embodiments, provided herein are methods of selecting a treatment course of action for a subject suffering from HTG, comprising: (a) characterizing hypertriglyceridemia (HTG) in a subject according to the methods described herein; and (b) administering, prescribing, or recommending one of the following treatment courses of action based on the results of step (a): (1) change to the lifestyle, diet, and/or medications of said subject if the subject does not suffer from HTG resulting from an apoprotein deficiency; (2) LpL-gene therapy if said subject suffers from HTG resulting from an LpL deficiency; (3) AV peptide therapy (See, e.g., U.S. Provisional App. No. 62/082,902; herein incorporated by reference in its entirety) and/or apoC-III antisense therapy if said subject suffers from HTG resulting from an apoprotein deficiency; (4) AV peptide therapy if said subject suffers from HTG resulting from an apoC-II LpL response deficiency; (5) CII peptide therapy if said subject suffers from HTG resulting from a apoA-V LpL response deficiency; (6) CII peptide, AV peptide, and/or apoC-III antisense therapy if said subject suffers from HTG resulting from overactive apoC-III; and (7) LpL-gene therapy and/or AV peptide therapy if said subject suffers from HTG resulting from multiple causes.

In some embodiments, provided herein are systems and/or kits comprising: reagents for measuring lipoprotein lipase (LpL) activity, apoC-II, apoA-V, and reference LpL. In some embodiments, the reagents for measuring LpL activity comprises fluorogenic-triglyceride (e.g., a triglyceride analog comprising a terminal fluorophore on one or more of the fatty acid chains).

In some embodiments, provided herein are systems and/or devices comprising: (a) a first well or chamber comprising reagents for measuring lipoprotein lipase (LpL) activity; (b) a second well or chamber comprising apoC-II and reagents for measuring LpL activity; (c) a third well or chamber comprising apoA-V and reagents for measuring LpL activity; and (d) a fourth well or chamber comprising reference LpLs and regents for measuring LpL activity. In some embodiments, the regents for measuring LpL activity comprises fluorogenic-triglyceride (e.g., a triglyceride analog comprising a terminal fluorophore on one or more of the fatty acid chains).

In some embodiments, provided herein are methods of using a system and/or device described herein to analyze a blood sample from a subject comprising: (a) obtaining or receiving the blood sample from the subject; (b) processing the blood sample processed to separate plasma and/or triglyceride-rich lipoproteins from leukocytes; (c) introducing portions of the leukocytes into the first, second, and third wells or chambers; (d) introducing a portion of the plasma and/or triglyceride-rich lipoproteins into the fourth well or chamber; and (e) detecting the reagents for measuring LpL activity or a reaction product thereof in each well or chamber. In some embodiments, the regents for measuring LpL activity comprise fluorogenic-triglyceride, and step (e) comprises detecting the fluorescent product of LpL hydrolysis of the fluorogenic-triglyceride.

In some embodiments, provided herein are devices, kits, and/or systems for performing a lipase hydrolytic potential assays and/or lipase stimulation response assays, comprising one or more wells, chambers, containers, or vessels comprising fluorogenic triglyceride substrate therein. In some embodiments, devices, kits, and/or systems comprise a microplate having fluorogenic triglyceride substrate (e.g., a triglyceride analog having a terminal fluorophore on one or more fatty acids) coated onto a surface of one or more wells therein. In some embodiments, the fluorogenic triglyceride substrate is dried onto a bottom surface of the well. In some embodiments, provided herein are methods of using a device, kit, or system to perform an assay comprising: (a) adding a sample comprising LpL to one or more of the wells, chambers, containers, or vessels; and (b) detecting fluorescence, wherein increased fluorescence upon addition of the sample correlates with LpL hydrolytic activity. In some embodiments, provided herein are methods of using a device, kit, or system to perform an assay comprising: (a) adding a sample comprising LpL to one or more of the wells, chambers, containers, or vessels; (b) adding an assay reagent comprising one or more apolipoproteins (e.g., apoC-II, apoA-V, etc.) or active peptide fragments thereof to the one or more of the wells, chambers, containers, or vessels; and (c) detecting fluorescence, wherein increased fluorescence upon addition of the sample correlates with LpL hydrolytic activity.

In some embodiments, provided herein are devices, kits, and/or systems for performing a lipase stimulating potential assay, comprising one or more wells, chambers, containers, or vessels comprising therein: (i) a fluorogenic triglyceride substrate and (ii) exogenous lipoprotein lipase (LpL). In some embodiments, the device, kit, or system comprises a microplate having fluorogenic triglyceride substrate (e.g., a triglyceride analog haing a terminal fluorophore on one or more fatty acids) and exogenous LpL coated onto a surface of one or more wells therein. In some embodiments, the fluorogenic triglyceride substrate and exogenous LpL is dried onto a bottom surface of the well. In some embodiments, provided herein are methods of using a device, kit, or system to perform a lipase stimulating potential assay comprising: (a) adding a sample comprising apolipoprotein from a subject to one or more of the wells, chambers, containers, or vessels; and (b) detecting fluorescence, wherein increased fluorescence upon addition of the sample compared with a control well without apolipoprotein indicates stimulation of LpL hydrolytic activity by a subject's apolipoprotein.

In some embodiments, provided herein are devices, kits, and/or systems comprising: (a) a triglyceride substrate, wherein the triglyceride substrate undergoes a detectable change upon hydrolysis by LpL; (b) an assay reagent comprising apoC-II or an active peptide fragment thereof; (c) an assay reagent comprising apoA-V or an active peptide fragment thereof; (d) an assay reagent comprising LpL. In some embodiments, the triglyceride substrate comprises fluorogenic-triglyceride (e.g., a triglyceride analog having a terminal fluorophore on one or more fatty acids).

In some embodiments, provided herein are devices, kits, and/or systems comprising: (a) a first well, chamber, container, or vessel comprising a triglyceride substrate, wherein the triglyceride substrate undergoes a detectable change upon hydrolysis by LpL; (b) a second well, chamber, container, or vessel comprising apoC-II or an active peptide fragment thereof, and the triglyceride substrate; (c) a third well, chamber, container, or vessel comprising apoA-V or an active peptide fragment thereof, and the triglyceride substrate; and (d) a fourth well, chamber, container, or vessel comprising reference LpL and the triglyceride substrate. In some embodiments, the triglyceride substrate comprises a fluorogenic-triglyceride (e.g., a triglyceride analog having a terminal fluorophore on one or more fatty acids). In some embodiments, provided herein are methods of using the device, kit, or system to analyze a blood sample from a subject comprising: (a) obtaining or receiving the blood sample from the subject; (b) processing the blood sample to separate plasma and/or triglyceride-rich lipoproteins from leukocytes; (c) introducing the leukocytes into the first, second, and third wells, chambers, containers, or vessels; (d) introducing a portion of the plasma and/or triglyceride-rich lipoproteins into the fourth well, chamber, container, or vessel; and (e) detecting the triglyceride substrate in each of the wells, chambers, containers, or vessels. In some embodiments, the triglyceride substrate comprises fluorogenic-triglyceride, and step (e) comprises detecting the fluorescent product of LpL hydrolysis of the fluorogenic-triglyceride.

In some embodiments, provided herein are reaction mixtures comprising: (a) reagents for measuring lipoprotein lipase (LpL) activity and leukocytes isolated from a blood sample; (b) purified apoC-II, reagents for measuring LpL activity, and leukocytes isolated from a blood sample; (c) purified apoA-V, reagents for measuring LpL activity, and leukocytes isolated from a blood sample; or (d) reference LpL, reagents for measuring LpL activity, and plasma and/or triglyceride-rich lipoproteins. In some embodiments, the regents for measuring LpL activity comprises fluorogenic-triglyceride (e.g., a triglyceride analog comprising a terminal fluorophore on one or more of the fatty acid chains). In some embodiments, provided herein is a set of reaction mixtures comprising two, three four or more reaction mixtures described herein (e.g., (a), (b), (c), and/or (d), above).

In some embodiments, provided herein are reaction mixtures comprising: (a) a detectable triglyceride substrate, buffer, and leukocytes isolated from a blood sample; (b) apoC-II or an active peptide fragment thereof, buffer, the detectable triglyceride substrate, and leukocytes isolated from a blood sample; (c) apoA-V or an active peptide fragment thereof, the detectable triglyceride substrate, and leukocytes isolated from a blood sample; or (d) LpL, the detectable triglyceride substrate, buffer, and plasma and/or triglyceride-rich lipoproteins. In some embodiments, the triglyceride substrate comprises fluorogenic-triglyceride (e.g., a triglyceride analog comprising a terminal fluorophore on one or more of the fatty acid chains). In some embodiments, provided herein are devices, kits, and/or systems comprising 2 or more (e.g., 2, 3, 4) of reaction mixtures of (a), (b), (c), and/or (d).

In some embodiments, systems, kits, and/or devices are provided for carrying out multiple steps of the methods (e.g., diagnostic assays) described herein. For example, a microplate (e.g., with sealed wells) is provided in which the wells contain some or all the necessary reagents for performing one or more (e.g., all) the steps of the assays described herein. In some embodiments, reagents are provided in a dried form (e.g., dried to the bottom of the wells, for reconstitution (e.g., in water or buffer)).

### BRIEF DESCRIPTION OF THE DRAWINGS

FIG. 1. Lipoprotein lipase (LpL) assay using a fluorogenic TG analog, (ENZCHEK) to determine LpL enzyme activity in a microplate format with product quantitation (fluorescence, λex=482 nm, λem=515 nm) in real-time (A). BODIPY-FA is used to determine the product concentration generated by the lipase (B). Substrate purchased from Invitrogen Inc.; ENZCHEK (C).

FIG. 2. The lipase assay is non-toxic and detects enzyme activity with live cells. The removal of the reaction mix from the cells (HepG2) stops substrate hydrolysis but is reinitiated once added back to the same cells (A). Washing cells with heparin inhibits substrate hydrolysis (B).

FIG. 3. Diagnostic assay schematic. Plasma and leukocytes are collected from a standard blood sample. 1) Lipase stimulating potential of whole plasma or TG-rich lipoproteins (TRLs) isolated with PEG-8000 are added (5-10% final) to a standard LpL preparation and the influence on enzymatic active determined. 2) Lipase hydrolytic potential of blood leukocytes is determined by incubating cells in the LpL reaction mix. Dashed line represents baseline (normal) levels.

FIG. 4. Plasma from patients with diabetes and dyslipidemia inhibit LpL activity. Plasma from healthy and diabetic individuals (de-identified from Zen-Bio Inc.) was added to standard lipase assay mix and incubated for 10 min at 37° C. (A). Normalizing the RFU to LpL without plasma (equals 1), the area under the curves can be used to calculate the lipase stimulating coefficient (B).

FIG. 5. apoB containing TRLs from diabetic patients inhibit LpL enzyme activity. TRLs were isolated with PEG-8000 from plasma and tested for its influence on LpL activity; 104 of TRLs or TRLs-depleted plasma (-TRLs) was added to standard LpL reaction mix and incu-bated for 10 min at 37° C.

FIG. 6. Leukocyte-LpL activity is detectible and responsive to C-II51-79 and AV199-232 peptide stimulation. Leukocytes (buffy coat) was collected by centrifugation of fresh whole blood from healthy donors (Zen Bio Inc.), rinsed and then incubated with LpL reaction mix (A, B). Leukocytes were also incubated with 50 μm CII51-79 and/or AV199-232 (A). Leukocyte LpL activity is detectable with small blood volumes (gain 130%) and substrate hydrolysis was lost after washing cells with heparin removing surface LpL (B). Increasing fluorescent plate reader gain can increase sensitivity to low femto molar range (C).

FIG. 7. Exemplary setup for performing (1) lipase stimulating potential, (2) lipase hydrolytic potential, and (3) lipase stimulation response assays on a single microplate.

FIG. 8. Lipase reaction reagents pre-dried onto microplates. To test the feasibility of drying down lipase assay reagents (LpL and fluorogenic (ENZCHEK) substrate) for later reconstitution, LpL and fluorogenic (ENZCHEK) substrate were reformulated and dried down in microplate wells. Determining the lipase hydrolytic potential (LHP) of THP-1 monocytic cells using standard (Fresh sub.) and pre-dried substrate (Dried sub.) (A) The fluorogenic (ENZCHEK) substrate was dissolved to 50 μM in ethanol and 44 was added to the microplate wells and the plate was then dried under vacuum at room temperature for 2 hrs (Dried sub.). To start the reaction, 50 μL of 20 mM Tris-HCl, 150 mM NaCl, 1%(w/v) BSA, 0.03%(w/v) zwittergent 3-14, pH 7.8 (TBS-BSA-ZWT) was added to each well to dissolve the fluorogenic (ENZCHEK) substrate . Live cells (THP-1) were then resuspended in 50 μL of 20 mM Tris-HCl, 150 mM NaCl, 1%(w/v) BSA, pH 7.8 (TBS-BSA) and added to the wells containing the dissolved substrate and incubated for 20 min at 37° C. Determining the activity of pre-dried LpL (B) To microplate wells, 44 of EnzChek substrate was added (as in part A), followed by 2 μL of LpL (not mixed with EnzChek), diluted 1:50 in 10 mM ammonium acetate pH 7.0 and then dried down under vacuum. To start the enzyme reaction, 50 μL of TBS-BSA-ZWT was added to the wells followed by 50 μL of TBS-BSA and reaction was monitored for 16-min at room temperature. The reaction product generated was assayed by fluorescent (λex=482 nm, λem=515 nm).

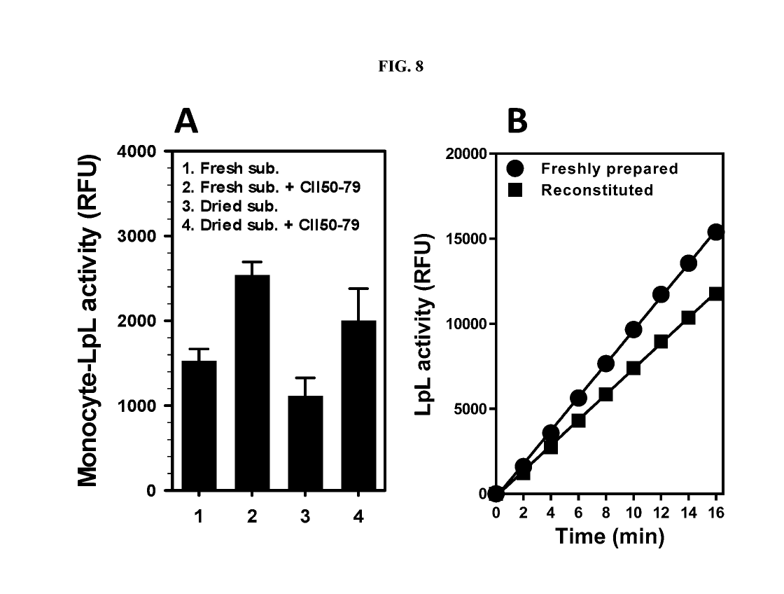

### DEFINITIONS

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise. Thus, for example, reference to “a triglyceride substrate” is a reference to one or more triglyceride substrates and equivalents thereof known to those skilled in the art, and so forth.

Many embodiments herein are described using open “comprising” language. Such embodiments encompass multiple closed “consisting of” and/or “consisting essentially of” embodiments, which may alternatively be claimed or described using such language.

As used herein, the term “subject” broadly refers to any animal, including but not limited to, human and non-human animals (e.g., dogs, cats, cows, horses, sheep, poultry, fish, crustaceans, etc.). As used herein, the term “patient” typically refers to a subject that is being treated for a disease or condition.

### DETAILED DESCRIPTION

Provided herein are systems (e.g., reagents, devices, etc.) and methods for characterization of hypertriglyceridemia (HTG) in a subject. In particular, systems and methods, are provided for identifying the specific deficiency(ies) leading to HTG, and selecting an appropriate strategy for the treatment of HTG based thereon.

For TG to be cleared from circulation it is first be hydrolyzed into free fatty acids (FFA) and mono-acylglycerol that are taken up and utilized by the cell. The overall rate of hydrolysis and removal of TG from the plasma is dependent on both cell surface and plasma components. The hydrolysis of TG is performed by cell surface lipoprotein lipase (LpL) and its TG-hydrolytic activity is in turn regulated primarily by specific apolipoproteins that are bound to TRLs. The best characterized of these are apoC-II and apoA-V, which are stimulators of LpL activity (lowering TG) and apoA-II, apoC-I and apoC-III, which are inhibitors of LpL activity (increasing TG). In addition, the serum protein angiopoietin like-4 (ANGPTL4) is a known inhibitor of LpL. Consequently when LpL associates with TRLs, the rate of TG hydrolysis and clearance from plasma is controlled by the balance between inhibitory and stimulatory proteins present in circulation.

The importance of the apolipoproteins in HTG is underscored by genome wide association studies (GWAS) that have identified the APOA1/C3/A4/A5 gene cluster (chromosome 11q23) as an important determinant of dylipidemia. Severe forms of HTG (TG>1000 mg/dL) are often the result of inherited loss-of-function mutations (familial hyperlipidemia) involving LpL (Fredrickson classification type Ia and V) or apoC-II (Fredrickson classification type Ib). In addition to the complete loss-of-function mutations, many sequence variants have also been reported that result in only a partial but physiologically significant loss-of-function. For LpL over 50 gene variants have been identified that negatively impact enzyme activity and TG levels (uniprot.org/uniprot/P06858). The most common LpL variant (Asn291Ser) is present in 4-6% of the population (ref. 6; herein incorporated by reference in its entirety) and causes a 25-50% loss of catalytic activity (ref. 7; herein incorporated by reference in its entirety).

HTG is commonly detected when a routine lipid profile is obtained to screen for cardiovascular risk. Often LDL and HDL cholesterol/TG levels may also be determined. Lipoprotein classes may also be assessed by electrophoresis and/or ultracentrifugation to determine lipoprotein patterns; however, this analysis is expensive and not available at many hospital laboratories. There is no standard clinical assay available to measure disease related changes in lipoprotein function and genetic testing for apolipoproteins and LpL variants is not commonly performed. The determination of the underlying cause of HTG is an important factor in the selection of appropriate treatment regimens. For patients with moderately elevated TG (499-200 mg/dL) physicians attempt to first identify potential secondary causes for the HTG, such as lifestyle, diet or certain medications, followed by more involved assessments for diabetes, kidney and liver function. More severe cases of HTG (>500 mg/dL) may draw attention to potential metabolic deficiencies that may involve the LpL or LpL-regulatory plasma apolipoproteins.

Clinicians can attempt to evaluate LpL activity in blood samples following the injection of heparin to release the enzyme from the cell surface. Post-heparin LpL assays have been used clinically since the 1940s (ref. 8; herein incorporated by reference in its entirety); however, its diagnostic value is uncertain since in recent studies the method was found to be unreliable in identifying genetically defective LpL in well characterized dyslipidemic populations (refs. 9,10; herein incorporated by reference in their entireties). Other limitations of post-heparin lipase assays include: (1) requiring additional steps to neutralize other related lipases (endothelial lipase and hepatic lipase) that are also released by heparin, (2) the inability to differentiate between inactive LpL, which is normally released into the circulation over physiologically active cell associated enzyme, (3) heparin has been reported to both stimulate and inhibit LpL activity, and (4) the risk of hemorrhagic complications inherent with administering a potent anticoagulant (ref. 11; herein incorporated by reference in its entirety).

In some embodiments, provided herein are lipase assay systems that analyze the activity of endogenous cell-associated LpL from a blood sample, without the need for prior heparin injection or the neutralization of other lipases that would normally be released by heparin. In some embodiments, assays systems measure the LpL stimulating potential of specific apolipoproteins present in blood samples. In some embodiments, by assessing the activity of cell-associated LpL as well as the LPL stimulating potential of apolipoproteins, assay systems provide a comprehensive characterization of the TG clearance pathway/capacity of a subject.

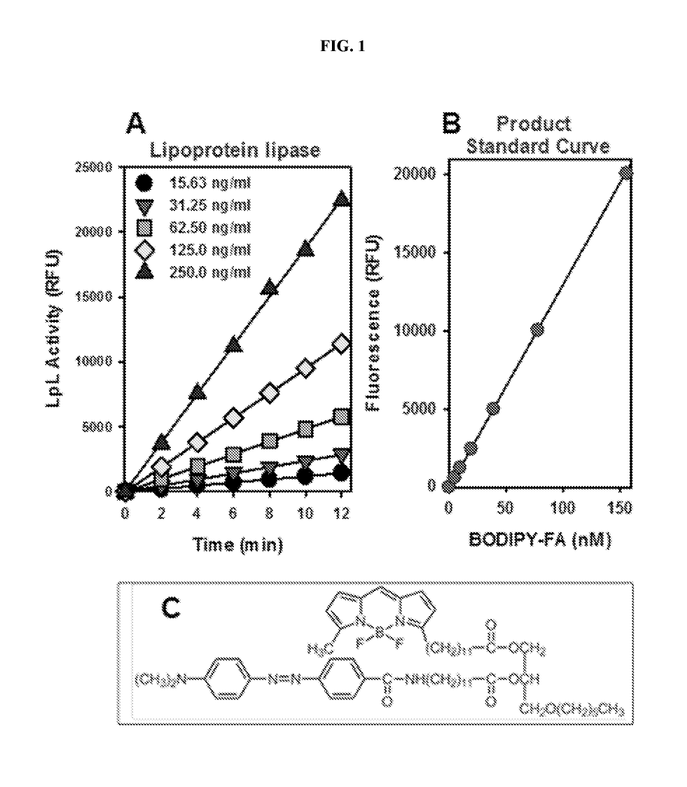

In some embodiments, systems and methods herein utilize a fluorescence-based lipase assay for clinical applications. In some embodiments, to assess lipase activity and the influence of its regulatory factors, a robust fluorescence-base assay is provided. Some embodiments of the assay utilize a fluorogenic-TG substrate (e.g., a triglyceride analog comprising a terminal fluorophore on one or more of the fatty acid chains (e.g., ENZCHEK, Life Technologies Inc.)), which generates a green-fluorescent product that does not require purification from the reaction mix prior to detection (FIG. 1). 1,2-o-dilauryl-rac-glycero-3-glutaric acid-(6′-methyl-resorufin) ester (DGGR) has also been utilized in similar assays.

In some embodiments, assays herein utilize a TG substrate that undergoes a change in one or more detectable characteristics upon being hydrolyzed by LpL. In some embodiments, the TG substrate that undergoes a quantifiable change in one or more detectable characteristics upon being hydrolyzed by LpL. In some embodiments, the degree of hydrolysis of the TG substrate is monitored in real-time and/or determined at one or more timepoints. In some embodiments, a TG substrate comprises a fluorogenic or colormetric substrate that undergoes and optically-detectable change upon hydrolysis by LpL. For example, the color or intensity of the TG substrate changes, increases, decreases, etc. upon hydrolysis. In particular embodiments, a TG substrate is a fluorogenic substrate (e.g., a TG analog that exhibits a change in fluorescence (e.g., intensity, emission wavelength, etc.) upon hydrolysis by LpL). In some embodiments, a fluorogenic TG substrate comprises a triglyceride scaffold (e.g., glycerol and three fatty acids) with a fluorophore linked thereto. In some embodiments, a fluorophore is linked to one or more (e.g., 1, 2, 3) of the fatty acids on a TG substrate. Suitable fluorophore include, for example: BODIPY, coumarin, N-hydroxysuccinimide (NHS) modified coumarin and succinimide or sulfonosuccinimide modified BODIPY, rhodamine (R110, rhodols, CRG6, Texas Methyl Red (TAMRA), Rox5, FAM, or fluorescein), coumarin derivatives (e.g., 7 aminocoumarin, and 7-hydroxycoumarin, 2-amino-4-methoxynapthalene, 1-hydroxypyrene, resorufin, phenalenones or benzphenalenones (U.S. Pat. No. 4,812,409)), acridinones (U.S. Pat. No. 4,810,636), anthracenes, and derivatives of alpha and beta-naphthol, fluorinated xanthene derivatives including fluorinated fluoresceins and rhodols (e.g., U.S. Pat. No. 6,162,931), etc. Embodiments herein are not limited by the fluorophores that find use in embodiments herein. In some embodiments, the fluorophore is attached to the terminus of one of the three fatty acids of the TG substrate. Exemplary fluorogenic TG substrates include, for example:

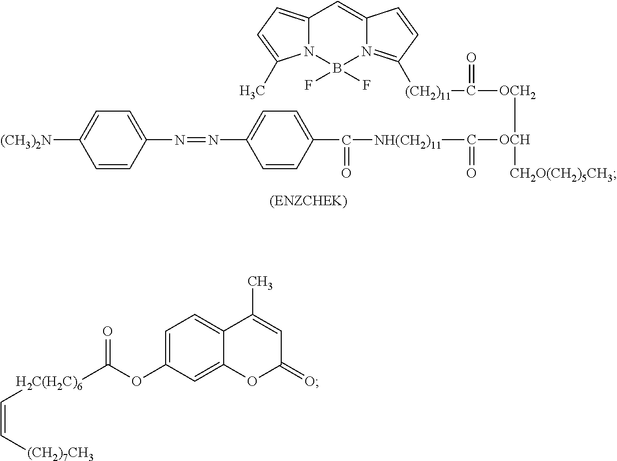

and 1,2-dioleoyl-3-pyrenedecanoyl-rac-glycerol in n-butanol. Embodiments herein are not limited by the TG-substrates that find use in embodiments herein. In some embodiments, a TG-substrates exhibits no fluorescence, background fluorescence, or reduced fluorescence prior to interaction with an active lipase (e.g., LpL), but exhibits a detectable increase in fluorescence upon hydrolysis. In some embodiments, the increase in fluorescence is measure against a control value. In some embodiments, the increase in fluorescence is monitored in real-time.

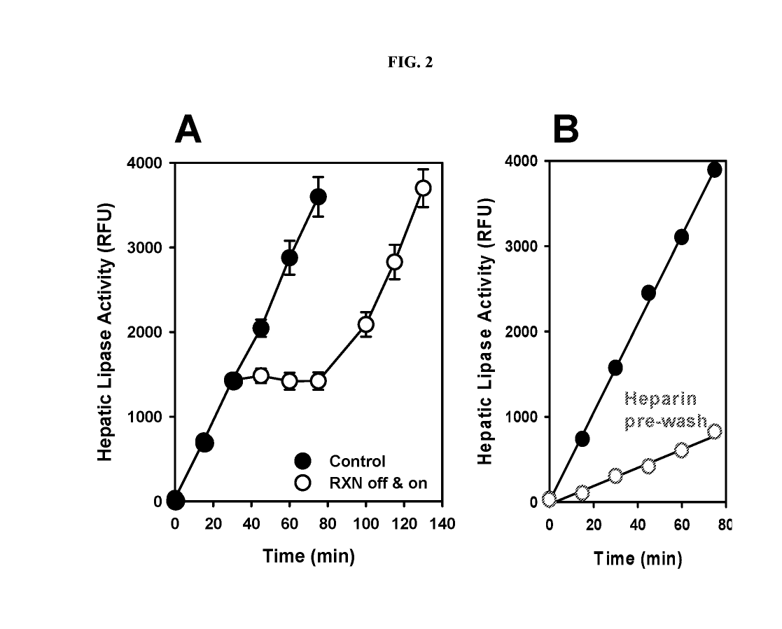

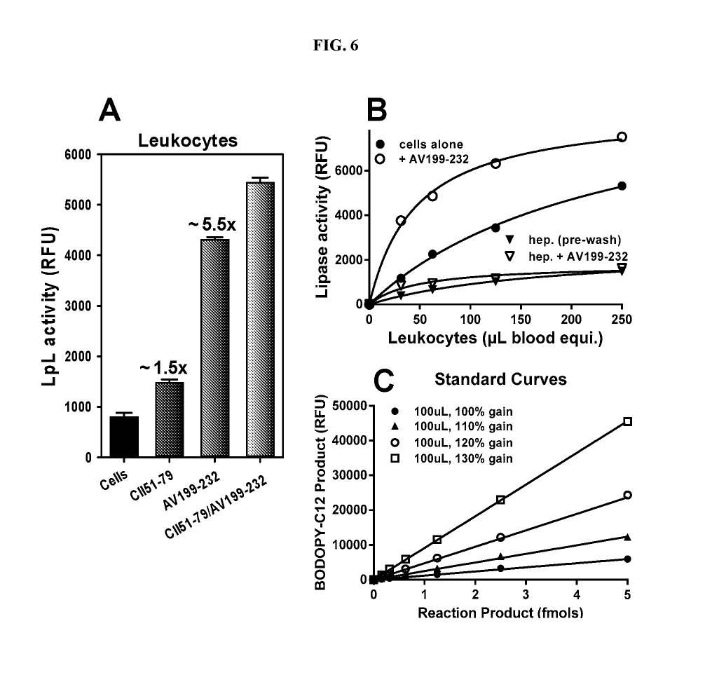

Experiments were conducted during development of embodiments of the present invention using an in vitro assay that utilizes bovine milk LpL (SIGMA) diluted to ˜120 ng/ml in Tris-HCl, 0.15M NaCl (TBS), 1%(w/v) BSA, 0.015%(w/v) Zwittergent 3-14, pH 7.8+1 μM to 25 μM ENZCHEK substrate, prepared in black polypropylene microplates. In some embodiments, the lipase reaction is performed at room temperature or 37° C. for 10 min to 60 min. Detection of product is performed with a fluorescent plate reader using excitation/emission wavelengths of 482 nm and 515 nm. The assay is sensitive to fmol/L of LpL (130% gain on reader) (FIG. 6C) and product accumulation increases linearly with increasing enzyme concentrations (FIG. 1A). The absolute product concentration is proportional to the relative fluorescence units (RFU) and can be calculated using BODIPY-C12 fatty acid (D3823, Life Technologies Inc.) as a standard and allows the determination of reaction rates (i.e. ng/ml/min) and Michaelis-Menten kinetic parameters. In some embodiments, the lipase assays is performed in two basic configurations to determine: (i) relative reaction rates at a fixed low concentrations of substrate, and/or (ii) enzyme kinetics using a range of substrate concentrations to acquire Michaelis-Menten kinetics constants (Vₘₐₓ & Kₘ). Furthermore, the mild nature of the assay formulation allows LpL activity to be determined with live cells in cell culture or isolated cells (FIG. 2).

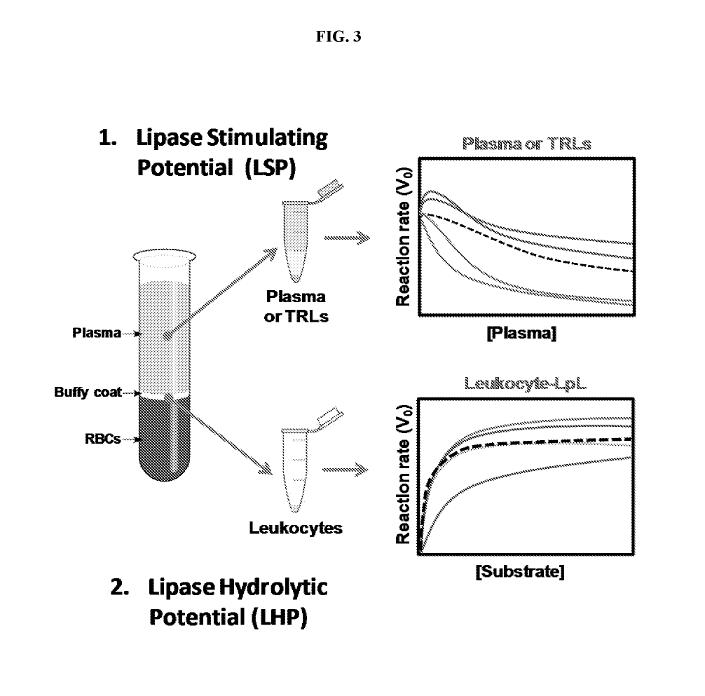

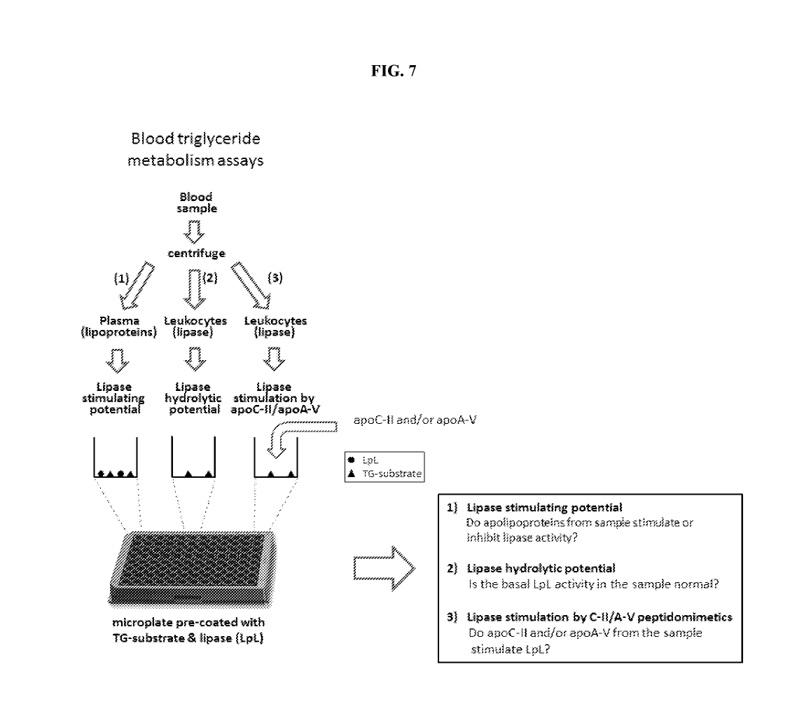

In some embodiments, systems and methods herein provide assays (e.g., companion assays) for the diagnosis of HTG, determining the underlying cause of HTG, and/or identifying a suitable treatment course of action for an individual diagnosed with HTG. Provided herein is a set of blood tests to rapidly screen for deficiencies in the TG clearance pathway in patients with HTG. In some embodiments, assays utilize a single low-volume blood sample that is analyzed using fluorescence-based assays in a microplate format. Blood samples may be processed prior to analysis. For example, in some embodiments, samples are first fractionated by centrifugation to collect the plasma (or serum) and the leukocytes (white blood cells) that are then assayed in at least two separate reactions (FIG. 3): (1) plasma proteins are assayed for LpL stimulating potential (LSP), and (2) leukocytes are assayed for endogenous LpL hydrolytic potential (LHP). In some embodiments, leukocytes are also assayed for LpL response to stimulation by apolipoproteins (FIG. 7).

In some embodiments, the data generated from the assays described herein provide for identification of patients with deficiencies in: (i) LpL activity, (ii) apolipoprotein function and/or, (iii) other factors in plasma. In some embodiments, assays herein allow for clinicians to forgo additional, more expensive and invasive testing that would otherwise be performed with patients. In addition, assays described herein are capable of rapid identification of patients that would benefit from genetic testing and indicate which target gene(s) to investigate (e.g., LpL, apolipoproteins, etc.). In some embodiments, assays require only a standard blood sample collected using standard techniques and without prior pre-injection of heparin (e.g., to dislodge cell surface LpL). The plasma and leukocytes (white blood cells in “buffy coat”) are then isolated by bench top density-gradient centrifugation by standard techniques and analyzed separately. In some embodiments, samples are frozen and stored for later analysis (or shipped).

To analyze the plasma's influence on LpL hydrolytic activity, plasma collected by traditional methods is analyzed whole or further fractionated into lipoproteins (e.g., TG-rich lipoproteins (TRLs)) and lipoprotein-depleted fractions (plasma/serum proteins). Lipoproteins are isolated by either precipitation with the addition of polyethylene glycol-8000 (PEG-8000) or by density centrifugation, which separates the different lipoprotein classes based on their densities. Both methods are well established procedures.

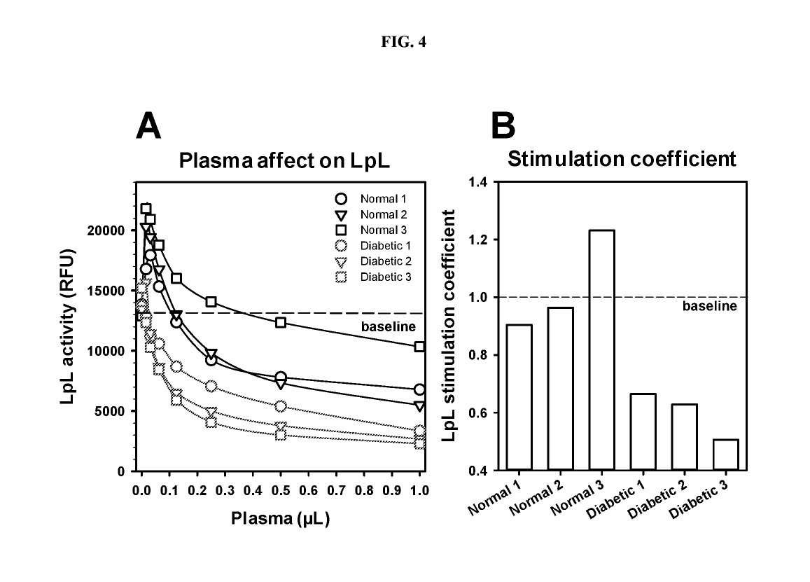

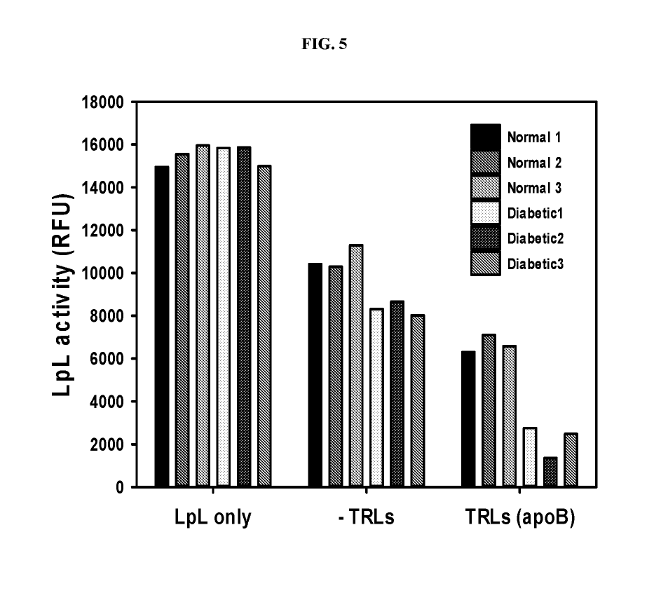

In some embodiments, whole plasma (FIG. 4A) isolated apoB containing TRLs prepared by 5.7% PEG-8000 precipitation (FIG. 5) or TRL-depleted plasma are added to standard LpL preparations to measure the influence of patient plasma components on lipase activity. Lipoprotein classes may also be isolated by a more involved density centrifugation method. In one configuration of the assay, standard LpL preparations are incubated with serially diluted plasma samples and the RFU (relative fluorescence units) versus plasma volume plotted. The assay reveals both inhibitory (apoC-III) and stimulatory components (apoC-II, apoA-V) of the plasma at the higher and lower concentrations, respectively. In some embodiments, the area under the resulting curves are normalized (LpL without plasma equals 1) to generate a lipase stimulating coefficient of plasma (FIG. 4B). These assays indicate if the combination of apolipoproteins or other plasma factors present in the patient's blood inhibit LpL activity and contributing to the impaired clearance of TG.

In some embodiments, systems and methods provide for characterization of endogenous LpL hydrolytic potential (LHP) of patient leukocytes. In some embodiments, systems and methods provide for characterization of the hydrolytic potential of patient LpLs. In some embodiments, a fluorescence-based lipase assay is used that is non-toxic to cells and hence can be used to detect cell-associate LpL activity in live cells. Assays detect the LpL activity of leukocytes isolated from small volumes of blood (e.g., 0.1-2 mL) and for healthy individuals, leukocyte LpL activity is responsive to both apoC-II and apoA-V peptide stimulation (FIG. 6).

In some embodiments, leukocytes (buffy coat) isolated by centrifugation of whole blood are washed with reaction buffer and then resuspended to 20,000 to 100,000 cells in 100 μL reaction buffer+ENZCHEK TG substrate. After incubation (10-60 min) the enzyme reactions are quantified (RFU) without the need to separate the cells from the reaction mix. A standard amount of apoC-II and apoA-V, or their corresponding peptide derivatives (CII50-79 and AV199-232) (FIG. 6A) can be added to the reaction to determine if the patient's LpL responds normally to these physiological stimulators (lipase response to stimulation assay). To establish the specific activity of LpL and the degree of LpL deficiency due perhaps to specific mutations, the amount of LpL in the patient samples is determined, for example, by commercially available ELISA kits, to acquire the specific activity (pmol/ug LpL) of the LpL. Lipases with catalytic deficiencies present with below normal specific activities.

In some embodiments, the assays described herein provide patient data to support clinicians in diagnosis and/or characterization of HTG. In some embodiments, methods herein guide the selection of appropriate treatments, or additional genetic and other testing, based on the outcomes of a suite of diagnostic assays (Table 1). In some embodiments, systems and methods characterize the underlying cause(s) of HTG, rather than its mere presence. In some embodiments, assays and systems provide diagnosis and/or characterization early is the disease process, and limit needless exploratory procedures that incur significant expense and patient risk. In some embodiments, assays use only a small blood sample and are performed under standard clinical laboratory setting.

TABLE 1
Assay results and diagnostic indications. Patient leukocytes-LpL and
plasma/TRLs assayed in separate reactions. The importance deficiencies
that can be identified; i) LpL deficiency LpL (low basal activity or poor
responsiveness to apoC-II and apoA-V stimulation) and, ii) deficiency
in patient plasma activators (apoC-II, apoA-V) or overactive inhibitors
(apoC-I, apoC-III).

| Patient leukocyte | | Patient | | |
|---|---|---|---|---|
| LpL activity | | plasma/TRLs | | |
| Basal | Stimulation by | stimulation of | Diagnosis | |
| Activity | apoC-II/apoA-V | reference LpL | of HTG | Potential therapy |
| + | by both | + | normal | lifestyle/diet/ |
| | | | | medications |
| − | Partially | + | LpL | LpL-gene therapy |
| | | | deficiency | |
| + | by both | − | apoprotein | AV peptide/ |
| | | | deficiency | apoC-III |
| | | | | antisense |
| + | not by apoC-II | + | LpL | AV-peptide |
| | | | response | |
| | | | deficiency | |
| + | not by apoA-V | + | LpL | CIII-peptides |
| | | | response | |
| | | | deficiency | |
| + | apoC-II/A-V | + | overactive | CII/AV-peptide, |
| | normal | | apoC-III | apoC-III antisense |
| − | partially | − | mixted | LpL-gene thereapy/ |
| | | | | AV-peptide |

In some embodiments, information determined by the systems and methods herein includes, but is not limited to: (1) LpL hydrolytic activity (e.g., basal enzyme activity (specific activity) and responsiveness to natural stimulators (e.g., apoC-II and apoA-V), etc.) and (2) identification of plasma factors that over-inhibit (e.e.g, apoC-III, ANGPTL4, or other plasma factors) or under-stimulate (apoC-II or apoA-V) LpL activity.

In some embodiments, systems, devices, and regents are provided for performing the assays described herein. As described, in some embodiments, systems and methods herein utilize a microplate-based fluorometric assay. Accordingly, in some embodiments microplates are provided for performing the assays described herein. In some embodiments, microplates are provided with assay reagents immobilized, coated, dried, and/or within one or more wells of the microplate. In some embodiments, wells are provided (and labeled) for performing multiple assays described herein (e.g., hydrolytic activity, stimulating potential, response to stimulation, etc.). In some embodiments, wells are provided for use as controls. In some embodiments, wells are pre-loaded with all or a portion of the appropriate reagents for performing assays. In some embodiments, wells are pre-loaded with TG substrate, TG substrate and LpL (e.g., in a manner to prevent premature reaction), TG-substrate and apolipoproteins, etc. In some embodiments, reagents are provided separately from a microplate (e.g., a microplate may be supplied by a user). In some embodiments, assays are performed in a vessel or container other than a microplate (e.g., tube, capillary, etc.).

In some embodiments, to perform an assay an appropriate sample (e.g., plasma, leukocytes, etc.) is added to a reaction vessel (e.g., well of a microplate). In some embodiments, the sample is diluted in buffer prior to adding to the reaction vessel. In some embodiments, buffer is used to dissolve reagents coated, immobilized, and/or dried onto the inside of a reaction vessel. Any suitable buffer may find use in embodiments herein. For example, phosphate buffers, HEPES, MOPS, HEPPS, and Tris-acetate, glycine, etc. Embodiments are not limited to the use of these specific buffers. Other components that may find use in reagent mixtures include surfactants, EDTA, DTT, metal cations (e.g. magnesium (Mg⁺²)), etc.

In some embodiments, fluorescence is measured or monitored using a fluorometer or plate reader. In some embodiments, fluorescence intensity in a fluorescence microplate reader equipped with standard filters may be measured at a single time point (e.g., 30 sec, 1 min, 2 min, 5 min, 10 min, 15 min, 30 min, 1 hr, or ranges therebetween) over multiple time points, or in real time. The plate can be read in any of a number of fluorescent plate readers. These readers have, for example, a light source which is directed from above the plate and the resultant fluorescence is detected by a detector positioned either directly above the plate or at an angle above the plate. Other plate readers and or fluorometers are also within the scope herein.

In some embodiments, active peptide fragments of apolipoproteins, or apolipoprotein mimetics find use in the assays described herein or in treatments indicated by the assays described herein. Examples of such peptides and mimetics of apolipoproteins are described in, for example, International Application No. PCT/US2015/061845; incorporated by reference in its entirety.

### REFERENCES

The following references, some of which are cited above by number (ref.X), are herein incorporated by reference in their entireties.
1. Ford, E. S., Li, C., Zhao, G., Pearson, W. S., Mokdad, A. H. (2009) Hypertriglyceridemia and its pharmacologic treatment among US adults. Arch.Intern.Med. 169, 572-578
2. Christian, J. B., Bourgeois, N., Snipes, R., Lowe, K. A. (2011) Prevalence of severe (500 to 2,000 mg/dl) hypertriglyceridemia in United States adults. Am.J.Cardiol. 107, 891-897
3. Khetarpal, S. A., Rader, D. J. (2015) Triglyceride-rich lipoproteins and coronary artery disease risk: new insights from human genetics. Arterioscler.Thromb.Vasc.Biol. 35, e3-e9
4. Do, R., et al. (2013) Common variants associated with plasma triglycerides and risk for coronary artery disease. Nat.Genet. 45, 1345-1352
5. Fredrickson, D. S., Lees, R. S. (1965) A SYSTEM FOR PHENOTYPING HYPERLIPOPROTEINEMIA. Circulation 31, 321-327
6. Gerdes, C., Fisher, R. M., Nicaud, V., Boer, J., Humphries, S. E., Talmud, P. J., Faergeman, O. (1997) Lipoprotein lipase variants D9N and N291S are associated with increased plasma triglyceride and lower high-density lipoprotein cholesterol concentrations: studies in the fasting and postprandial states: the European Atherosclerosis Research Studies. Circulation 96, 733-740
7. Reymer, P. W., Gagne, E., Groenemeyer, B. E., Zhang, H., Forsyth, I., Jansen, H., Seidell, J. C., Kromhout, D., Lie, K. E., Kastelein, J., . (1995) A lipoprotein lipase mutation (Asn291Ser) is associated with reduced HDL cholesterol levels in premature atherosclerosis. Nat.Genet. 10, 28-34
8. Hahn, P. F. (1943) Abolishment of Alimentary Lipemia Following Injection of Heparin. Science 98, 19-20
9. Mero, N., Suurinkeroinen, L., Syvanne, M., Knudsen, P., Yki-Jarvinen, H., Taskinen, M. R. (1999) Delayed clearance of postprandial large TG-rich particles in normolipidemic carriers of LPL Asn291Ser gene variant. J.Lipid Res. 40, 1663-1670
10. van, H. M., Dallinga-Thie, G. M., Steyerberg, E. W., Sijbrands, E. J. (2009) Diagnostic value of post-heparin lipase testing in detecting common genetic variants in the LPL and LIPC genes. Eur.J.Hum.Genet. 17, 1386-1393
11. Ellis, M. H., Hadari, R., Tchuvrero, N., Shapira, S., Kovlenko, I., Kozmiakova, M., Zissin, R., Elis, A. (2006) Hemorrhagic complications in patients treated with anticoagulant doses of a low molecular weight heparin (enoxaparin) in routine hospital practice. Clin.Appl.Thromb.Hemost. 12, 199-204
12. Basu, D., Manjur, J., Jin, W. (2011) Determination of lipoprotein lipase activity using a novel fluorescent lipase assay. J.Lipid Res. 52, 826-832

1. A method of assessing lipoprotein lipase (LpL) activity in a subject, comprising:
(a) exposing a blood sample from the subject to a triglyceride substrate, wherein the triglyceride substrate undergoes a detectable change upon hydrolysis by LpL; and
(b) detecting the hydrolysis of the triglyceride substrate.

2. The method of claim 1, wherein the triglyceride substrate is a fluorescent or colormetric triglyceride substrate which undergoes a change in fluorescence or color upon hydrolysis by LpL.

3. The method of claim 2, wherein the triglyceride substrate is a fluorogenic triglyceride substrate.

4. The method of claim 3, wherein the fluorogenic triglyceride substrate exhibits increased fluorescence upon hydrolysis by LpL.

5. The method of claim 4, wherein the fluorogenic triglyceride substrate is a quenched substrate.

6. The method of claim 1, wherein the LpL is circulating blood LpL.

7. The method of claim 6, wherein the LpL does not comprise endothelial-bound LpL.

8. The method of claim 1, wherein the blood sample and/or the subject have not been treated with heparin.

9. The method of claim 1, wherein the blood sample is whole blood, a leukocyte-containing fraction of a fractioned blood sample, or purified leukocytes.

10. A method of assessing lipoprotein lipase (LpL) activity in a subject, comprising:
(a) exposing a sample from the subject to a fluorogenic triglyceride substrate, wherein the fluorogenic triglyceride substrate undergoes a detectable increase in fluorescence intensity upon hydrolysis by LpL; and
(b) detecting fluorescence.

11. The method of claim 10, wherein the sample is an un-heparinized blood sample.

12. The method of claim 11, wherein the sample comprises a blood fraction comprising leukocytes.

13. The method of claim 10, wherein the LpL is circulating blood LpL.

14. The method of claim 13, wherein the LpL does not comprise endothelial-bound LpL.

15. The method of claim 10, wherein detecting fluorescence comprises measuring the fluorescence at a single timepoint.

16. The method of claim 10, wherein detecting fluorescence comprises monitoring fluorescence over time.

17. A method of assessing the responsiveness of lipoprotein lipase (LpL) in a subject to apolipoproteins, comprising:
(a) exposing a sample from the subject to a exogenous apolipoprotein; and
(b) detecting hydrolysis of a triglyceride substrate.

18. The method of claim 17, wherein the triglyceride substrate is a fluorescent or colormetric triglyceride substrate which undergoes a change in fluorescence or color upon hydrolysis by LpL.

19. The method of claim 18, wherein the triglyceride substrate is a fluorogenic triglyceride substrate.

20. The method of claim 19, wherein the fluorogenic triglyceride substrate exhibits increased fluorescence upon hydrolysis by LpL.

21. The method of claim 20, wherein the fluorogenic triglyceride substrate is a quenched substrate.

22. The method of claim 17, wherein the exogenous apolipoprotein comprises a known amount of an assay reagent comprising one or more apolipoproteins.

23. The method of claim 22, wherein the exogenous apolipoprotein comprises apoC-II or an active peptide fragment thereof.

24. The method of claim 22, wherein the exogenous apolipoprotein comprises apoA-V or an active peptide fragment thereof.

25. The method of claim 17, wherein detecting fluorescence comprises measuring the fluorescence at a single timepoint.

26. The method of claim 17, wherein detecting fluorescence comprises monitoring fluorescence over time.

27. The method of claim 17, wherein the LpL is circulating blood LpL.

28. The method of claim 27, wherein the LpL does not comprise endothelial-bound LpL.

29. The method of claim 17, wherein the sample is a blood sample selected from the group consisting of whole blood, a leukocyte-containing fraction of a fractioned blood sample, or purified leukocytes.

30. The method of claim 29, wherein the sample and/or the subject have not been treated with heparin.

31. The method of claim 17, further comprising comparing hydrolysis of the triglyceride substrate in the presence of exogenous apolipoprotein to a control value obtained in the absence of exogenous apolipoprotein.

32. A device, kit, or system for performing a method of one of claims 1-31 comprising one or more wells, chambers, containers, or vessels comprising fluorogenic triglyceride substrate therein.

33. The device, kit, or system of claim 32, comprising a microplate having fluorogenic triglyceride substrate coated onto a surface of one or more wells therein.

34. The device, kit, or system of claim 33, wherein the fluorogenic triglyceride substrate is dried onto a bottom surface of the well.

35. A method of using a device, kit, or system of one of claim 32-34 to perform an assay comprising:
(a) adding a sample comprising LpL to one or more of the wells, chambers, containers, or vessels; and
(b) detecting fluorescence, wherein increased fluorescence upon addition of the sample correlates with LpL hydrolytic activity.

36. A method of assessing the stimulating potential of a subject's apolipoprotein, comprising:
(a) exposing a sample from the subject to a exogenous lipoprotein lipase (LpL) and a triglyceride substrate; and
(b) detecting hydrolysis of the triglyceride substrate, wherein increased hydrolysis compared to a control of LpL and the triglyceride substrate indicates stimulation of LpL by the subject's apolipoproteins.

37. The method of claim 36, wherein the sample comprises a blood sample selected from the group consisting of whole plasma, fractionated plasma, and isolated lipoproteins.

38. The method of claim 37, wherein the sample does not comprise LpL.

39. The method of claim 36, wherein the triglyceride substrate is a fluorescent or colormetric triglyceride substrate which undergoes a change in fluorescence or color upon hydrolysis by LpL.

40. The method of claim 39, wherein the triglyceride substrate is a fluorogenic triglyceride substrate.

41. The method of claim 40, wherein the fluorogenic triglyceride substrate exhibits increased fluorescence upon hydrolysis by LpL.

42. The method of claim 41, wherein the fluorogenic triglyceride substrate is a quenched substrate.

43. A device, kit, or system for performing a method of one of claims 36-42 comprising one or more wells, chambers, containers, or vessels comprising therein: (i) a fluorogenic triglyceride substrate and (ii) exogenous lipoprotein lipase (LpL).

44. The device, kit, or system of claim 43, comprising a microplate having fluorogenic triglyceride substrate and exogenous LpL coated onto a surface of one or more wells therein.

45. The device, kit, or system of claim 44, wherein the fluorogenic triglyceride substrate and exogenous LpL is dried onto a bottom surface of the well.

46. A method of using a device, kit, or system of one of claim 43-45 to perform an assay comprising:
(a) adding a sample comprising apolipoprotein from a subject to one or more of the wells, chambers, containers, or vessels; and
(b) detecting fluorescence, wherein increased fluorescence upon addition of the sample compared with a control well without apolipoprotein indicates stimulation of LpL hydrolytic activity by a subject's apolipoprotein.

47. A method of assessing lipoprotein lipase (LpL) activity in a subject comprising:
(a) measuring basal lipoprotein lipase (LpL) activity by:
(i) exposing a blood sample from the subject to a triglyceride substrate, wherein the triglyceride substrate undergoes a detectable change upon hydrolysis by LpL; and
(ii) detecting the hydrolysis of the triglyceride substrate.

48. A method, comprising:
(a) obtaining a blood sample from a subject; and
(b) assaying two or more aspects of triglyceride hydrolysis, comprising:
(i) measuring basal lipoprotein lipase (LpL) activity in leukocytes from the sample;
(ii) contacting leukocytes from the sample with apoC-II or an active peptide fragment thereof, and measuring the effect on LpL activity;
(iii) contacting leukocytes from the sample with apoA-V or an active peptide fragment thereof, and measuring the effect on LpL activity; and/or
(iv) measuring the LpL stimulating potential of plasma and/or triglyceride-rich lipoproteins (TRLs) obtained from the sample.

49. The method of claim 48, comprising separating plasma and/or TRLs from the leukocytes in the sample.

50. The method of claim 48, wherein the LpL activity and the LpL stimulating potential are measured by a quantitative fluorescence assay system.

51. A method of characterizing hypertriglyceridemia (HTG) in a subject, comprising:
(a) performing the method of claim 48; and
(b) classifying or reporting the subject according to one or more of the following criteria:
(i) the subject does not suffer from HTG resulting from an apolipoprotein deficiency, LpL deficiency, or LpL response deficiency if:
(A) said leukocytes exhibit normal basal LpL activity,
(B) said leukocytes exhibit stimulation of LpL activity by apoC-II and apoA-V, and
(C) said whole plasma and/or triglyceride-rich lipoproteins exhibit LpL stimulating potential;
(ii) the subject suffers from HTG resulting from an LpL deficiency if:
(A) said leukocytes exhibit less than normal basal LpL activity,
(B) said leukocytes exhibit partial stimulation of LpL activity by apoC-II and/or apoA-V, and
(C) said whole plasma and/or triglyceride-rich lipoproteins exhibit LpL stimulating potential;
(iii) the subject suffers from HTG resulting from an apoprotein deficiency if:
(A) said leukocytes exhibit normal basal LpL activity,
(B) said leukocytes exhibit stimulation of LpL activity by apoC-II and apoA-V, and
(C) said whole plasma and/or triglyceride-rich lipoproteins do not exhibit LpL stimulating potential;
(iv) the subject suffers from HTG resulting from an LpL response deficiency if:
(A) said leukocytes exhibit normal basal LpL activity,
(B) said leukocytes exhibit stimulation of LpL activity by apoA-V, and
(C) said whole plasma and/or triglyceride-rich lipoproteins exhibit LpL stimulating potential;
(v) the subject suffers from HTG resulting from an LpL response deficiency if:
(A) said leukocytes exhibit basal LpL activity,
(B) said leukocytes exhibit stimulation of LpL activity by apoC-II, and
(C) said whole plasma and/or triglyceride-rich lipoproteins exhibit LpL stimulating potential;
(vi) the subject suffers from HTG resulting from overactive apoC-III if:
(A) said leukocytes exhibit normal basal LpL activity,
(B) said leukocytes do not exhibit stimulation of LpL activity by apoC-II or apoA-V, and
(C) said whole plasma and/or triglyceride-rich lipoproteins exhibit LpL stimulating potential; and
(vii) the subject suffers from HTG resulting from multiple causes if:
(A) said leukocytes exhibit less than normal basal LpL activity,
(B) said leukocytes exhibit partial stimulation of LpL activity by apoC-II and apoA-V, and
(C) said whole plasma and/or triglyceride-rich lipoproteins do not exhibit LpL stimulating potential.

52. A method of selecting a treatment course of action for a subject suffering from HTG, comprising:
(a) performing the steps of claim 51;
(b) prescribing or recommending one of the following treatment courses of action based on the results of step (a):
(1) change to the lifestyle, diet, and/or medications of said subject if the subject does not suffer from HTG resulting from an apoprotein deficiency;
(2) LpL-gene therapy if said subject suffers from HTG resulting from an LpL deficiency;
(3) AV peptide therapy and/or apoC-III antisense therapy if said subject suffers from HTG resulting from an apoprotein deficiency;
(4) AV peptide therapy if said subject suffers from HTG resulting from an apoC-II LpL response deficiency;
(5) CII peptide therapy if said subject suffers from HTG resulting from a apoA-V LpL response deficiency;
(6) CII peptide, AV peptide, and/or apoC-III antisense therapy if said subject suffers from HTG resulting from overactive apoC-III; and
(7) LpL-gene therapy and/or AV peptide therapy if said subject suffers from HTG resulting from multiple causes.

53. A device, kit, or system comprising:
(a) a triglyceride substrate, wherein the triglyceride substrate undergoes a detectable change upon hydrolysis by LpL;
(b) an assay reagent comprising apoC-II or an active peptide fragment thereof;
(c) an assay reagent comprising apoA-V or an active peptide fragment thereof;
(d) an assay reagent comprising LpL.

54. The device, kit, or system of claim 53, wherein the triglyceride substrate comprises fluorogenic-triglyceride.

55. A device, kit, or system comprising:
(a) a first well, chamber, container, or vessel comprising a triglyceride substrate, wherein the triglyceride substrate undergoes a detectable change upon hydrolysis by LpL;
(b) a second well, chamber, container, or vessel comprising apoC-II or an active peptide fragment thereof, and the triglyceride substrate;
(c) a third well, chamber, container, or vessel comprising apoA-V or an active peptide fragment thereof, and the triglyceride substrate; and
(d) a fourth well, chamber, container, or vessel comprising reference LpL and the triglyceride substrate.

56. The device, kit, or system of claim 55, wherein the triglyceride substrate comprises fluorogenic-triglyceride.

57. A method of using the device, kit, or system of claim 55 to analyze a blood sample from a subject comprising:
(a) obtaining or receiving the blood sample from the subject;
(b) processing the blood sample to separate plasma and/or triglyceride-rich lipoproteins from leukocytes;
(c) introducing the leukocytes into the first, second, and third wells, chambers, containers, or vessels;
(d) introducing a portion of the plasma and/or triglyceride-rich lipoproteins into the fourth well, chamber, container, or vessel; and
(e) detecting the triglyceride substrate in each of the wells, chambers, containers, or vessels.

58. The method of claim 57, wherein the triglyceride substrate comprises fluorogenic-triglyceride, and step (e) comprises detecting the fluorescent product of LpL hydrolysis of the fluorogenic-triglyceride.

59. A reaction mixture comprising
(a) a detectable triglyceride substrate, buffer, and leukocytes isolated from a blood sample;
(b) apoC-II or an active peptide fragment thereof, buffer, the detectable triglyceride substrate, and leukocytes isolated from a blood sample;
(c) apoA-V or an active peptide fragment thereof, the detectable triglyceride substrate, and leukocytes isolated from a blood sample; or
(d) LpL, the detectable triglyceride substrate, buffer, and plasma and/or triglyceride-rich lipoproteins.

60. The reaction mixture of claim 59, wherein the triglyceride substrate comprises fluorogenic-triglyceride.

61. A device, kit, or system comprising 2 or more of the reaction mixtures of claim 59.
